(19) United States
(12) Patent Application Publication
(10) Pub. No.: US 2011/0098765 A1
(43) Pub. Date: Apr. 28, 2011

(54) DETECTING LEAD RELATED CONDITION DURING DELIVERY OF THERAPEUTIC ELECTRICAL SIGNALS

(75) Inventor: Amisha S. Patel, Maple Grove, MN (US)

(73) Assignee: Medtronic, Inc., Minneapolis, MN (US)

(21) Appl. No.: 12/606,891

(22) Filed: Oct. 27, 2009

(51) Int. Cl. A61N 1/08; A61N 1/362; A61N 1/39

(52) U.S. Cl. 607/8; 607/62; 607/28

(57) ABSTRACT

In general, the disclosure describes techniques for detecting lead related conditions, such as lead fractures or other lead integrity issues. As described herein, lead related conditions are identified by detecting delivered energy and electrical path impedance during delivery of a therapeutic electrical to a patient. If one or both of the delivered energy and impedance traverse respective thresholds, a lead related condition may exist. The energy delivered during the electrical signal may be compared to the amount of energy the electrical signal was programmed to deliver to determine if the delivered energy is less than a threshold percentage of the programmed energy of the electrical signal. The impedance of the electrical path through which the therapeutic electrical signal is delivered may be compared to a threshold impedance value to determine if the impedance detected during the electrical signal is greater than the threshold.

### TECHNICAL FIELD

The disclosure relates to medical devices and, more particularly, medical devices that include leads to sense electrical signals within a patient and/or deliver electrical signals to a patient.

### BACKGROUND

A variety of medical devices for delivering therapy and/or monitoring a physiological condition have been used clinically or proposed for clinical use in patients. Examples include medical devices that deliver therapy to and/or monitor conditions associated with the heart, muscle, nerve, brain, stomach or other organs or tissue. Some therapies include the delivery of electrical stimulation to such organs or tissues. Some medical devices may employ one or more elongated electrical leads carrying electrodes for the delivery of electrical stimulation to such organs or tissues, electrodes for sensing electrical signals within the patient, which may be generated by such organs or tissue, and/or other sensors for sensing physiological parameters of a patient.

Medical leads may be configured to allow electrodes or other sensors to be positioned at desired locations for delivery of electrical stimulation or sensing. For example, electrodes or sensors may be carried at a distal portion of a lead. A proximal portion of the lead may be coupled to a medical device housing, which may contain circuitry such as stimulation generation and/or sensing circuitry. In some cases, the medical leads and the medical device housing are implantable within the patient. Medical devices with a housing configured for implantation within the patient may be referred to as implantable medical devices.

Implantable cardiac pacemakers or cardioverter-defibrillators, for example, provide therapeutic electrical stimulation to the heart via electrodes carried by one or more implantable medical leads. The electrical stimulation may include signals such as pulses or shocks for pacing, cardioversion or defibrillation. In some cases, a medical device may sense intrinsic depolarizations of the heart, and control delivery of stimulation signals to the heart based on the sensed depolarizations. Upon detection of an abnormal rhythm, such as bradycardia, tachycardia or fibrillation, an appropriate electrical stimulation signal or signals may be delivered to restore or maintain a more normal rhythm. For example, in some cases, an implantable medical device may deliver pacing pulses to the heart of the patient upon detecting tachycardia or bradycardia, and deliver cardioversion or defibrillation shocks to the heart upon detecting tachycardia or fibrillation.

Implantable medical leads typically include a lead body containing one or more elongated electrical conductors that extend through the lead body from a connector assembly provided at a proximal lead end to one or more electrodes located at the distal lead end or elsewhere along the length of the lead body. The conductors connect stimulation and/or sensing circuitry within an associated implantable medical device housing to respective electrodes or sensors. Some electrodes may be used for both stimulation and sensing. Each electrical conductor is typically electrically isolated from other electrical conductors and is encased within an outer sheath that electrically insulates the lead conductors from body tissue and fluids.

Medical lead bodies implanted for cardiac applications tend to be continuously flexed by the beating of the heart. Other stresses may be applied to the lead body, including the conductors therein, during implantation or lead repositioning. Patient movement can cause the route traversed by the lead body to be constricted or otherwise altered, causing stresses on the lead body and conductors. In rare instances, such stresses may fracture a conductor within the lead body. The fracture may be continuously present, or may intermittently manifest as the lead flexes and moves.

Additionally, the electrical connection between medical device connector elements and the lead connector elements can be intermittently or continuously disrupted. For example, connection mechanisms, such as set screws, may be insufficiently tightened at the time of implantation, followed by a gradual loosening of the connection. Also, lead pins may not be completely inserted.

Lead fracture, disrupted connections, or other causes of short circuits or open circuits may be referred to, in general, as lead related conditions. In the case of cardiac leads, sensing of an intrinsic heart rhythm through a lead can be altered by lead related conditions. Furthermore, delivery of electrical stimulation can be impaired by lead related conditions. Identifying lead related conditions may be challenging, particularly in a clinic, hospital or operating room setting, due to the often intermittent nature of lead related conditions. Identification of lead related conditions may allow modifications of the stimulation therapy or sensing, or lead replacement.

### SUMMARY

In general, techniques for detecting lead related conditions, such as lead fractures or other lead integrity issues are disclosed. A processor, for example, detects the energy delivered to a patient during delivery of a therapeutic electrical signal via an electrical path to the patient, and the path impedance during delivery of the electrical signal to a patient. If at least one of the delivered energy and impedance traverse a threshold, the processor identifies a lead related condition.

In one example, a method includes delivering a therapeutic electrical signal to a patient via an electrical path including at least one medical lead, detecting a delivered energy and an impedance of the path during the delivery of the electrical signal, and identifying a lead related condition when at least one of the detected delivered energy and the detected impendence traverses a respective threshold.

In another example, a system includes a medical lead, a signal generator, and a processor. The signal generator is connected to the lead and configured to deliver a therapeutic electrical signal via an electrical path that includes the lead. The processor is configured to detect a delivered energy and an impedance of the path during delivery of a therapeutic electrical signal to a patient, and to identify a lead related condition when at least one of the detected delivered energy and the detected impendence traverses a respective threshold.

In one more example, a system includes means for delivering a therapeutic electrical signal to a patient via an electrical path, means for detecting a delivered energy and an impedance of the path during the delivery of the electrical signal, and means for identifying a lead related condition when at least one of the detected delivered energy and the detected impendence traverses a respective threshold.

In another example, A computer-readable medium includes instructions for causing a programmable processor to deliver a therapeutic electrical signal to a patient via an electrical path including at least one medical lead, detect a delivered energy and an impedance of the path during the delivery of the electrical signal, and identify a lead related condition when at least one of the detected delivered energy and the detected impendence traverses a respective threshold.

### BRIEF DESCRIPTION OF DRAWINGS

FIG. 1 is a conceptual drawing illustrating an example system that includes an implantable medical device (IMD) coupled to implantable medical leads.

FIG. 2 is a conceptual drawing illustrating the example IMD and leads of FIG. 1 in conjunction with a heart.

FIG. 3 is a conceptual drawing illustrating the example IMD of FIG. 1 coupled to a different configuration of implantable medical leads in conjunction with a heart.

FIG. 4 is a functional block diagram illustrating an example configuration of the IMD of FIG. 1.

FIG. 5 is a functional block diagram illustrating an example configuration of an external programmer that facilitates user communication with the IMD.

FIG. 6 is a block diagram illustrating an example system that includes an external device, such as a server, and one or more computing devices that are coupled to the IMD and programmer shown in FIG. 1 via a network.

FIG. 7 is a flow diagram of an example method of identifying a lead related condition.

### DETAILED DESCRIPTION

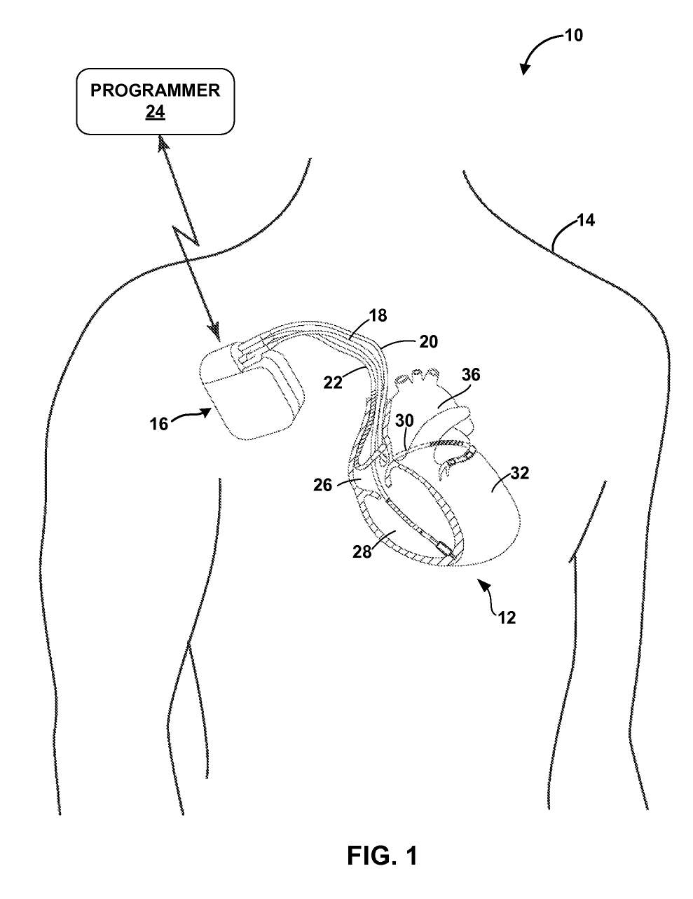

FIG. 1 is a conceptual drawing illustrating an example system 10 that may be used for sensing of physiological parameters of patient 14 and/or to provide therapy to heart 12 of patient 14. Therapy system 10 includes IMD 16, which is coupled to leads 18, 20, and 22, and programmer 24. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

In the following examples, techniques are described for detecting lead related conditions that may compromise the operation of a medical device, e.g., IMD 16. In the disclosed examples, a processor of a device, e.g., IMD 16 identifies lead related conditions by comparing one or more characteristics of the performance of IMD 16 during delivery of a therapeutic electrical signal to patient 14 to one or more thresholds. In particular, the energy delivered by and the lead impedance during delivery of a therapeutic electrical signal to patient 14 are detected by the processor and compared to a threshold, e.g., stored in a memory of IMD 16. If at least one of the delivered energy and impedance traverse a threshold, a lead related condition is identified. A therapeutic electrical signal, as used herein, generally means any electrical signal delivered to patient 14 that is configured to elicit a physiological response in the patient. Example therapeutic electrical signals include cardiac pacing pulses and high voltage defibrillation and cardioversion shocks.

Although an implantable medical device and delivery of electrical stimulation to heart 12 are described herein as examples, the techniques for detecting lead related conditions of this disclosure may be applicable to other medical devices and/or other therapies. In general, the techniques described in this disclosure may be implemented by any medical device, e.g., implantable or external, that includes leads to sense electrical signals within a patient and/or deliver electrical signals to a patient, or any components of a system including such a medical device. As one alternative example, IMD 16 may be a neurostimulator that delivers electrical stimulation to and/or monitor conditions associated with the brain, spinal cord, or neural tissue of patient 14.

In the example of FIG. 1, leads 18, 20, 22 extend into the heart 12 of patient 14 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into coronary sinus 30 to a region adjacent to the free wall of left ventricle 32 of heart 12. Right atrial (RA) lead 22 extends through one or more veins and the vena cava, and into the right atrium 26 of heart 12.

In some examples, therapy system 10 may additionally or alternatively include one or more leads or lead segments (not shown in FIG. 1) that deploy one or more electrodes within the vena cava or other vein. These electrodes may allow alternative electrical sensing configurations that may provide improved or supplemental sensing in some patients. Furthermore, in some examples, therapy system 10 may additionally or alternatively include temporary or permanent epicardial or subcutaneous leads, instead of or in addition to transvenous, intracardiac leads 18, 20 and 22. Such leads may be used for one or more of cardiac sensing, pacing, or cardioversion/defibrillation.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may detect arrhythmia of heart 12, such as tachycardia or fibrillation of ventricles 28 and 32, and may also provide defibrillation therapy and/or cardioversion therapy via electrodes located on at least one of the leads 18, 20, 22. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In some examples, programmer 24 comprises a handheld computing device, computer workstation, or networked computing device. Programmer 24 may include a user interface that receives input from a user. It should be noted that the user may also interact with programmer 24 remotely via a networked computing device.

A user, such as a physician, technician, surgeon, electrophysiologist, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD.

For example, the user may use programmer 24 to retrieve information from IMD 16 regarding the rhythm of heart 12, trends therein over time, or arrhythmic episodes. As another example, the user may use programmer 24 to retrieve information from IMD 16 regarding other sensed physiological parameters of patient 14, such as intracardiac or intravascular pressure, activity, posture, respiration, or thoracic impedance. As another example, the user may use programmer 24 to retrieve information from IMD 16 regarding the performance or integrity of IMD 16 or other components of system 10, such as leads 18, and 22, or a power source of IMD 16. In some examples, this information may be presented to the user as an alert. For example, a lead related condition identified based on the delivered energy and/or lead impedance during delivery of a therapeutic electrical signal to patient 14 may trigger IMD 16 to transmit an alert to the user via programmer 24.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

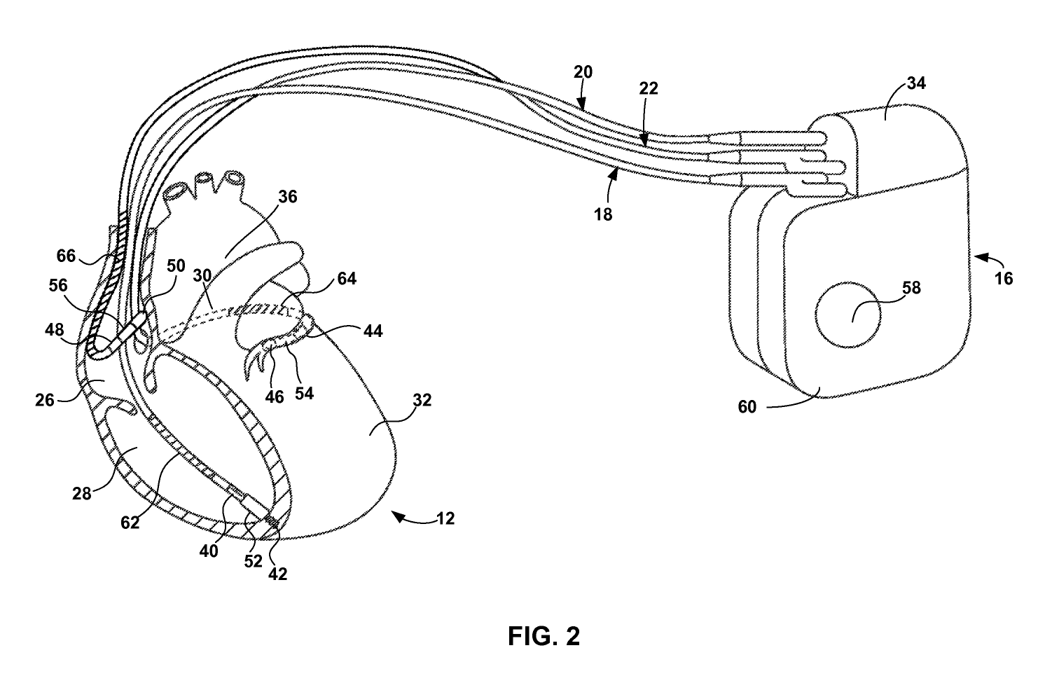

FIG. 2 is a conceptual diagram illustrating IMD 16 and leads 18, 20 and 22 of therapy system 10 in greater detail. Leads 18, 20, 22 may be electrically coupled to a signal generator, e.g., stimulation generator, and a sensing module of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44 and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

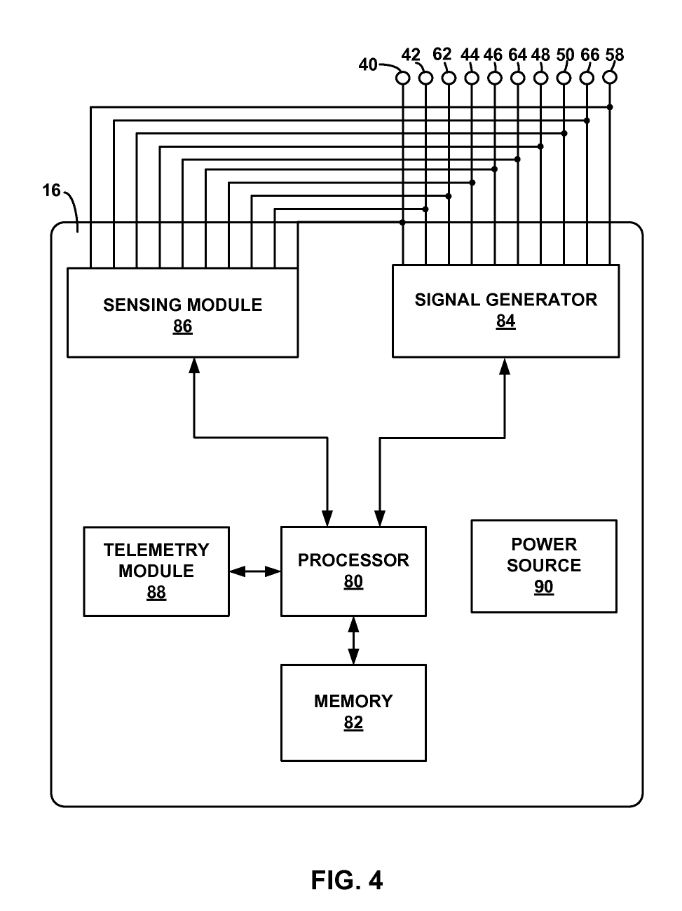

In some examples, as illustrated in FIG. 2, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 4, housing 60 may enclose a signal generator that generates therapeutic electrical signals, such as cardiac pacing pulses and defibrillation shocks, as well as a sensing module for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of stimulation or sensing, their associated conductors and connectors, and any tissue or fluid between the electrodes, may define an electrical path.

The configuration of therapy system 10 illustrated in FIGS. 1 and 2 is merely one example. In other examples, a therapy system may include epicardial leads and/or patch electrodes instead of or in addition to the transvenous leads 18, 20, 22 illustrated in FIG. 1. Further, IMD 16 need not be implanted within patient 14. In examples in which IMD 16 is not implanted in patient 14, IMD 16 may deliver defibrillation pulses and other therapies to heart 12 via percutaneous leads that extend through the skin of patient 14 to a variety of positions within or outside of heart 12.

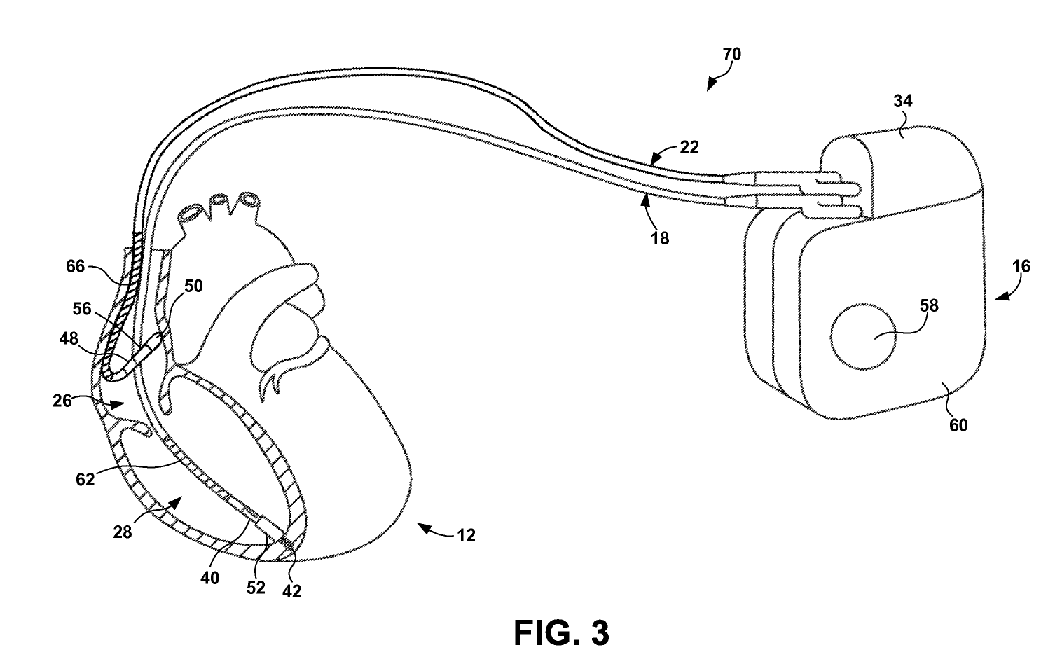

In addition, in other examples, a therapy system may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 36. As another example, other examples of therapy systems may include a single lead that extends from IMD 16 into right atrium 26 or right ventricle 28, or two leads that extend into a respective one of the right ventricle 26 and right atrium 26. An example of this type of therapy system is shown in FIG. 3. Any electrodes located on these additional leads may be used in sensing and/or stimulation configurations.

Additionally, as previously mentioned, IMD 16 need not deliver therapy to heart 12. In general, this disclosure may be applicable to any medical device, e.g., implantable or external, that includes leads to sense and/or deliver electrical signals to a patient.

FIG. 3 is a conceptual diagram illustrating another example of therapy system 70, which is similar to therapy system 10 of FIGS. 1 and 2, but includes two leads 18, 22, rather than three leads. Leads 18, 22 are implanted within right ventricle 28 and right atrium 26, respectively. Therapy system 70 shown in FIG. 3 may be useful for providing defibrillation and pacing pulses to heart 12. Detection of lead related conditions according to this disclosure may be performed in two lead systems in the manner described herein with respect to three lead systems.

FIG. 4 is a functional block diagram illustrating an example configuration of IMD 16. In the illustrated example, IMD 16 includes a processor 80, memory 82, signal generator 84, sensing module 86, telemetry module 88, and power source 90. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Processor 80 controls signal generator 84 to deliver stimulation therapy to heart 12 according to one or more selected therapy programs, which may be stored in memory 82. For example, processor 80 may control stimulation generator 84 to deliver electrical pulses with the amplitudes, pulse widths, frequency, or electrode polarities specified by the selected one or more therapy programs.

Signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, signal generator 84 is configured to generate and deliver electrical stimulation therapy to heart 12. For example, signal generator 84 may deliver defibrillation shocks to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Electrical sensing module 86 monitors signals from at least one of electrodes 40, 42, 44, 46, 48, 50, 58, 62, 64 or 66 in order to monitor electrical activity of heart 12. Sensing module 86 may also include a switch module to select which of the available electrodes are used to sense the heart activity, depending upon which electrode combination is used in the current sensing configuration. In some examples, processor 80 may select the electrodes that function as sense electrodes, i.e., select the sensing configuration, via the switch module within sensing module 86. Processor 80 may control the functionality of sensing module 86 by providing signals via a data/address bus.

Sensing module 86 may include one or more detection channels, each of which may comprise an amplifier. The detection channels may be used to sense cardiac signals. Some detection channels may detect events, such as R- or P-waves, and provide indications of the occurrences of such events to processor 80. One or more other detection channels may provide the signals to an analog-to-digital converter, for processing or analysis by processor 80. In response to the signals from processor 80, the switch module within sensing module 86 may couple selected electrodes to selected detection channels.

For example, sensing module 86 may comprise one or more narrow band channels, each of which may include a narrow band filtered sense-amplifier that compares the detected signal to a threshold. If the filtered and amplified signal is greater than the threshold, the narrow band channel indicates that a certain electrical cardiac event, e.g., depolarization, has occurred. Processor 80 then uses that detection in measuring frequencies of the sensed events. Different narrow band channels of sensing module 86 may have distinct functions. For example, some various narrow band channels may be used to sense either atrial or ventricular events.

In one example, at least one narrow band channel may include an R-wave amplifier that receives signals from the sensing configuration of electrodes 40 and 42, which are used for sensing and/or pacing in right ventricle 28 of heart 12. Another narrow band channel may include another R-wave amplifier that receives signals from the sensing configuration of electrodes 44 and 46, which are used for sensing and/or pacing proximate to left ventricle 32 of heart 12. In some examples, the R-wave amplifiers may take the form of an automatic gain controlled amplifier that provides an adjustable sensing threshold as a function of the measured R-wave amplitude of the heart rhythm.

In addition, in some examples, a narrow band channel may include a P-wave amplifier that receives signals from electrodes 48 and 50, which are used for pacing and sensing in right atrium 26 of heart 12. In some examples, the P-wave amplifier may take the form of an automatic gain controlled amplifier that provides an adjustable sensing threshold as a function of the measured P-wave amplitude of the heart rhythm. Other amplifiers may also be used. Furthermore, in some examples, one or more of the sensing channels of sensing module 86 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In some examples, sensing module 86 includes a wide band channel which may comprise an amplifier with a relatively wider pass band than the R-wave or P-wave amplifiers. Signals from the sensing electrodes that are selected for coupling to this wide-band amplifier may be converted to multi-bit digital signals by an analog-to-digital converter (ADC) provided by, for example, sensing module 86 or processor 80. In some examples, processor 80 may store the digitized versions of signals from the wide band channel in memory 82 as electrograms (EGMs). Processor 80 may employ digital signal analysis techniques to characterize the digitized signals from the wide band channel to, for example detect and classify the patient's heart rhythm. Processor 80 may detect and classify the patient's heart rhythm by employing any signal processing methodologies appropriate for the intended application or applications of IMD 16.

If IMD 16 is configured to generate and deliver pacing pulses to heart 12, processor 80 may maintain programmable counters which control the basic time intervals associated with single or multiple chamber pacing. Intervals defined by processor 80 may include atrial and ventricular pacing escape intervals, refractory periods during which sensed P-waves and R-waves are ineffective to restart timing of the escape intervals, and the pulse widths of the pacing pulses. The durations of these intervals may be determined by processor 80 in response to stored data in memory 82. Processor 80 may also determine the amplitude of the cardiac pacing pulses.

During pacing, processor 80 may reset escape interval counters upon sensing of R-waves and P-waves with detection channels of sensing module 86. Signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. Processor 80 may reset the escape interval counters upon the generation of pacing pulses by signal generator 84, and thereby control the basic timing of cardiac pacing functions, including anti-tachyarrhythmia pacing.

The value of the count present in the escape interval counters when reset by sensed R-waves and P-waves may be used by processor 80 to measure the durations of R-R intervals, P-P intervals, P-R intervals and R-P intervals, which are measurements that may be stored in memory 82. Processor 80 may use the count in the interval counters to detect a suspected tachyarrhythmia event, such as ventricular fibrillation or ventricular tachycardia. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies may also be employed by processor 80 in other examples.

In some examples, processor 80 may determine that tachyarrhythmia has occurred by identification of shortened R-R (or P-P) interval lengths. Generally, processor 80 detects tachycardia when the interval length falls below 220 milliseconds (ms) and fibrillation when the interval length falls below 180 ms. These interval lengths are merely examples, and a user may define the interval lengths as desired, which may then be stored within memory 82. In some examples, the interval length is detected for a certain number of consecutive cycles, for a certain percentage of cycles within a running window, or a running average for a certain number of cardiac cycles.

Processor 80 may also control sensing module 86 to identify lead related conditions. Detection of lead related conditions may prevent or end inappropriate detection of cardiac events. Rapid, intermittent fracture of one or more of leads 18, 20, 22 or disconnection of the lead from IMD 16 may be interpreted by the IMD 16 as a plurality of sensed cardiac events, e.g., R-waves, and result in inappropriate detection of a cardiac arrhythmia by IMD 16. Additionally, IMD 16 may be incapable of delivering or may deliver inadequate therapy to patient 14 in the event that one or more of leads 18, 20, 22 develop a fracture or are otherwise compromised.

In examples disclosed herein, therefore, processor 80 identifies lead related conditions by comparing one or more characteristics of the performance of IMD 16 during delivery of a therapeutic electrical signal to patient 14 to one or more thresholds. In particular, the energy delivered via an electrical path for the electrical signal to patient 14, and the impedance of the path, during delivery of the signal, are detected by processor 80 and compared to respective thresholds stored in memory 82. If at least one of the delivered energy and impedance traverse a threshold, a lead related condition is identified. The disclosed lead related condition identification techniques are, in general, automatically triggered by processor 80 any time IMD 16 delivers a therapeutic electrical signal to patient 14 in response to detection of an abnormal rhythm of heart 12. Examples of therapeutic electrical signals delivered by IMD 16 and during which lead related conditions may be identified include pacing pulses configured to capture cardiac tissue, as well as defibrillation and cardioversion shocks. In comparison to most pacing pulses, defibrillation and cardioversion shocks are high voltage signals including 10 times or more voltage than is delivered by an IMD during cardiac pacing.

In one example, sensing module 86 and/or processor 80 are capable of collecting, measuring, and/or calculating delivered energy and impedance data for any electrical path including two or more of electrodes 40, 42, 44, 46, 48, 50, 58, 62, or 66. In such examples, IMD 16 detects an abnormal rhythm in heart 12 and, in response thereto, processor 80 control signals generator 84 to deliver a therapeutic electrical signal, e.g. a shock to patient 14 between at least two electrodes that are coupled to one or more of leads 18, 20 and 22. Sensing module 86 and/or processor 80 detect delivered energy and impedance values during delivery of the signal between the electrodes. In particular, for example, during delivery of a defibrillation or cardioversion shock, sensing module 86 and/or processor 80 may collect, measure, and/or calculate delivered energy and impedance data based on delivery of the shock between two of coil electrodes 62, 64, 66 or based on delivery of the shock between one or more of electrodes 62, 64, 66 and housing electrode 58. Sensing module 86 and/or processor 80 may collect, measure, and/or calculate delivered energy and impedance values for any of a variety of electrical paths that include one or more electrodes on one or more of leads 18, 20, and 22 based on delivery of a therapeutic electrical signal between any combination of two or more of electrodes 40, 42, 44, 46 and 48, elongated electrodes 62, 64 and 66, and housing electrode 58. Processor 80 may store detected delivered energy and impedance values in memory 82.

In some examples, IMD 16 detects delivered energy and impedance by delivering, from stimulation generator 84, a therapeutic shock in the form of a high-voltage pulse between first and second electrodes, and measuring the actual current delivered through the lead to which the electrodes are connected during delivery of the shock. IMD 16, e.g., processor 80, calculates a resistance based upon the voltage amplitude of the pulse and the measured amplitude of the resulting current. Additionally, processor 80 calculates the actual energy delivered to patient 14 by the shock based on the voltage amplitude of the shock, the resulting current, and the time over which the shock was delivered.

In certain cases, IMD 16 detects delivered energy and impedance by delivering, from stimulation generator 84, a therapeutic shock in the form of a high-current pulse across first and second electrodes, and measuring the actual voltage delivered through the lead to which the electrodes are connected during delivery of the shock. Processor 80 of IMD 16 calculates a resistance based upon the current amplitude of the pulse and the measured amplitude of the resulting voltage. Additionally, processor 80 calculates the actual energy delivered to patient 14 by the shock based on the current amplitude of the shock, the resulting voltage, and the time over which the shock was delivered. In some examples, sensing module 86 of IMD 16 includes circuitry for measuring amplitudes of resulting currents or voltages, such as sample and hold circuitry.

In certain cases, IMD 16 may detect impedance values that include both a resistive and a reactive (i.e., phase) component. In such cases, IMD 16 may measure impedance during delivery of a sinusoidal or other time varying signal by signal generator 84, for example. Thus, as used herein, the term “impedance” is used in a broad sense to indicate any collected, measured, and/or calculated value that may include one or both of resistive and reactive components. Additionally, delivered energy and impedance data may include actual, measured values, or may include values that can be used to calculate delivered energy and/or impedance, such as current and/or voltage values. For example, a delivered energy may include a measured current or voltage value rather than an energy value derived from a known voltage or current and a measured current or voltage. In some examples, a delivered energy may comprise a voltage or current measured during delivery of a constant voltage pulse or a voltage or current measured during delivery of a constant current pulse.

To identify lead related conditions, processor 80 compares at least one of the detected energy delivered by and the impedance during delivery of the therapeutic electrical signal to patient 14 to a threshold. In some examples, processor 80 compares the actual energy delivered by the signal to patient 14 to a threshold percentage of a programmed energy of the signal stored in memory 82. Processor 80 of IMD 16, in general, is programmed to deliver therapy to patient 14 according to one or more programmed parameters organized as therapy programs and program groups and stored in memory 82. One such parameter may include the amount of energy a therapeutic electrical signal is intended to deliver to heart 12 of patient 14.

Although signal generator 84, if functioning properly, will generate and deliver the programmed energy in the form of a voltage or current pulse to the electrical path programmed for the therapeutic electrical signal, the energy actually delivered to patient 14 may be diminished in the event a lead related condition exists somewhere along the electrical path through which the signal is delivered. In order to identify a lead related condition, therefore, processor 80 may determine if the energy delivered to patient 14 by the signal is less than a percentage threshold of the programmed energy of the signal. In some examples, the threshold percentage of programmed energy is in a range from approximately 60% to approximately 80%. In one example, the threshold percentage of programmed energy is approximately equal to 75%.

In some examples, processor 80 compares the lead impedance during delivery of the therapeutic electrical signal to patient 14 to a threshold impedance value stored in memory 82. In general, it is understood that lead impedance values greater than 200 ohms are indicative of some type of lead related condition that may compromise the functions of IMD 16. In some examples disclosed herein, the threshold impedance value is in a range from approximately 180 ohms to approximately 220 ohms. In one example, the threshold impedance value is approximately equal to 200 ohms.

In the examples disclosed herein, processor 80 performs lead integrity testing automatically during the delivery of any therapeutic electrical signal to patient 14. If an integrity issue is detected along one electrical path through which the signal is delivered, processor 80 may test alternate electrode configurations to identify which conductor or connector of the path is experiencing an integrity issue. For example, if an integrity issue is detected when two of electrodes 62, 64, 66 are activated to deliver a defibrillation or cardioversion shock to patient 14, processor 80 may test any of electrodes 62, 64, 66 independently, e.g., by separately testing each of 62, 64, 66 in combination with housing electrode 58, to determine which one of electrodes 62, 64, 66 is causing the issue.

Processor 80 may take one or more actions in response to detecting a lead related condition. For example, processor 80 may reconfigure sensing and/or therapy delivery to avoid use of paths with integrity issues. Additionally or alternatively, processor 80 may reconfigure sensing and/or therapy delivery parameters for paths with integrity issues. As one example, processor 80 may select different combinations of electrodes to deliver therapy to patient 14. As another example, processor 80 may extend the blanking period of one or more sensing channels, e.g., amplifiers, of sensing module 86. In one more example, processor 80 may increase a sensing threshold, e.g., a threshold used to detect cardiac events, such as depolarizations, following delivery of a therapeutic electrical signal, e.g., an antitachycardia pacing pulse. Extending a blanking period and/or increasing a threshold value may help prevent inappropriate detection of arrhythmias and/or other cardiac events.

In addition to reconfiguring operation of various components of IMD 16, processor 80 may also provide an alert to a user, e.g., of programmer 24, regarding any detected lead related conditions via telemetry module 88. Additionally or alternatively, IMD 16 may suggest a response to a lead related condition and/or receive user approval of a response via telemetry module 88. Alternatively, IMD 16 may provide the delivered energy and/or impedance detected during the therapeutic electrical signal, or other sensed signal to an external device, e.g., programmer 24, via telemetry module 88 for confirmation of identification of lead related conditions. In some examples, processor 80 may provide detected energies or impedances in the form of a trend diagram illustrating the values over time.

Telemetry module 88 includes any suitable hardware, firmware, software or any combination thereof for communicating with another device, such as programmer 24 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

In some examples, processor 80 may transmit atrial and ventricular heart signals (e.g., electrocardiogram signals) produced by atrial and ventricular sense amp circuits within sensing module 86 to programmer 24. Programmer 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In addition, processor 80 may transmit information regarding lead related conditions to programmer 24 via telemetry module 88. For example, processor 80 may provide an alert regarding any detected lead related conditions, suggest a response to a lead related condition, or provide a delivered energy and/or impedance detected during delivery of a therapeutic electrical signal, or other sensed signal for identification of lead related conditions to programmer 24 via telemetry module 88. Processor 80 may also receive information regarding lead related conditions or responses to such conditions from programmer 24 via telemetry module 88.

In some examples, IMD 16 may communicate, via programmer 24 or another external device, with a network such as the Medtronic CareLink® Network developed by Medtronic, Inc., of Minneapolis, Minn., or some other network linking patient 14 to a clinician or other users. In such examples, IMD 16 may pass the alert through the network to such users.

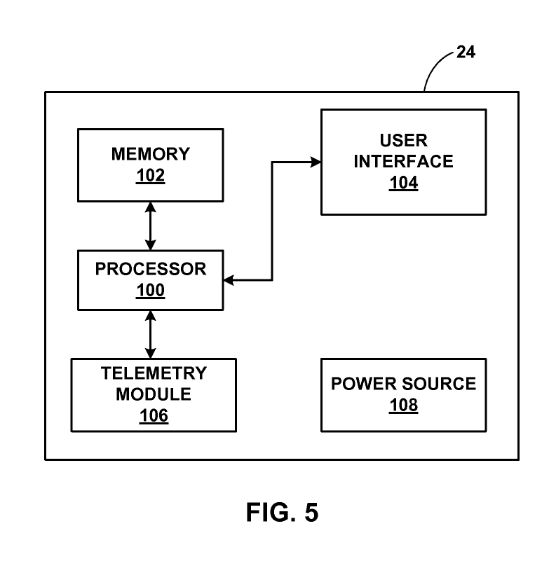

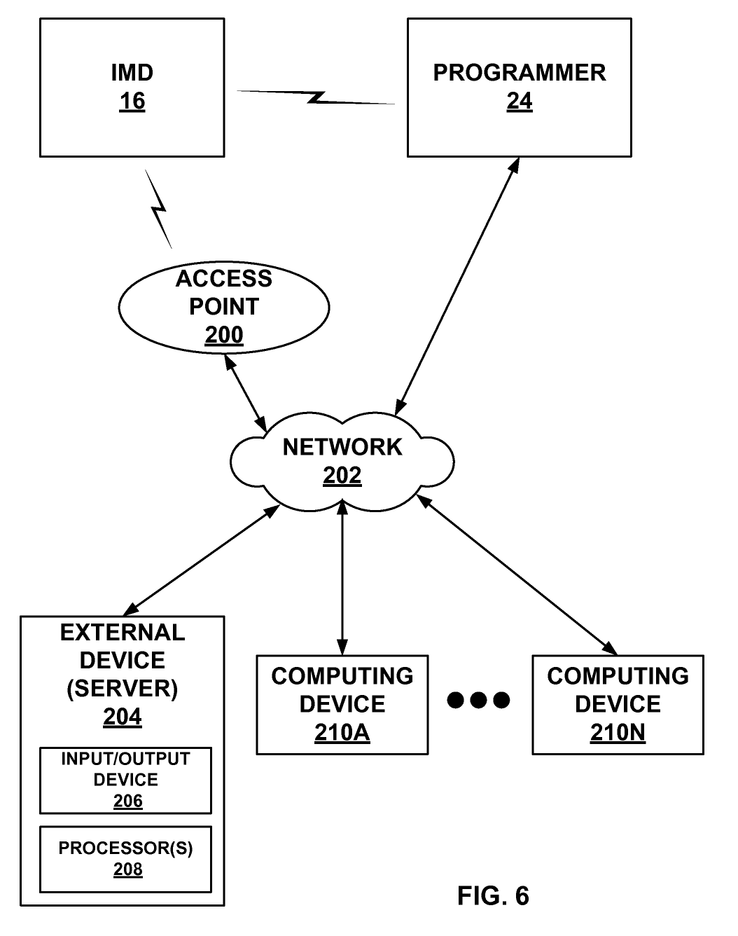

The functions for identifying lead related conditions attributed to processor 80 and memory 82 of IMD 16 may be implemented logically and/or physically as a separate module within IMD 16 or another device including, e.g., programmer 24. For example, IMD 16 may include a lead diagnostics unit including various modules for performing functions related to identifying lead related conditions for any of leads 18, 20, 22. Each module of the lead diagnostics unit may be implemented in one or more processors, such as processor 80 of IMD 16, processor 100 of programmer 24 (FIG. 5), and/or processor(s) 133 of external device 132 (FIG. 6). One or more modules of the lead diagnostics unit may additionally or alternatively be embodied in other digital or analog circuitry, such as sample and hold or other analog circuitry of sensing module 86. The modules of the lead diagnostics unit may be embodied as one or more hardware modules, software modules, firmware modules, or any combination thereof. As described herein with reference to processor 80, the lead diagnostics unit may automatically detect and analyze the energy delivered by and the lead impedance during the delivery of a therapeutic electrical signal to heart 12 of patient 14.

In one example, the diagnostic unit includes energy and impedance detection modules, a threshold comparison module, and an integrity indication module. Energy and impedance detection modules detect the delivered energy and impedance values for one or more electrical paths through which electrical signals are delivered by IMD 16. Threshold comparison module receives the delivered energy and lead impedance detected during delivery of the electrical signal and compare one or both to a threshold. For example, threshold comparison module receives the actual energy delivered by the electrical signal and compares the value to a threshold percentage of the energy the electrical signal was programmed to deliver by IMD 16. Threshold comparison module may also receive the detected lead impedance and compare the value to a threshold impedance value. In the event either or both of the delivered energy or impedance traverse a threshold, integrity indication module signals a lead related condition.

FIG. 5 is functional block diagram illustrating an example configuration of programmer 24. As shown in FIG. 5, programmer 24 may include a processor 100, memory 102, user interface 104, telemetry module 106, and power source 108. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 104, which may include display to present graphical user interface to a user, and a keypad or another mechanism for receiving input from a user.

The user may also use programmer 24 to adjust or control the detection of lead related conditions performed by IMD 16. For example, the user may use programmer 24 to program the thresholds to which delivered energy and lead impedance are compared, or any other aspects of the integrity test. In this manner, the user may be able to finely tune the integrity test to the specific condition of patient 14. In some examples, the user uses programmer 24 to control the performance of an integrity test for detecting lead related conditions, e.g., in a clinic, hospital, or operating room setting, at the time of implant or during a follow-up visit.

In addition, the user may receive an alert from IMD 16 indicating a potential lead related condition via programmer 24. The user may respond to IMD 16 by suggesting a response to a detected lead related condition. Alternatively, IMD 16 may automatically suggest a response to a lead related condition. Programmer 24 may prompt the user to confirm the response.

Processor 100 can take the form one or more microprocessors, DSPs, ASICs, FPGAs, programmable logic circuitry, or the like, and the functions attributed to processor 100 herein may be embodied as hardware, firmware, software or any combination thereof. Memory 102 may store instructions that cause processor 100 to provide the functionality ascribed to programmer 24 herein, and information used by processor 100 to provide the functionality ascribed to programmer 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient.

Programmer 24 may communicate wirelessly with IMD 16, such as using RF communication or proximal inductive interaction. This wireless communication is possible through the use of telemetry module 106, which may be coupled to an internal antenna or an external antenna. An external antenna that is coupled to programmer 24 may correspond to the programming head that may be placed over heart 12, as described above with reference to FIG. 1. Telemetry module 106 may be similar to telemetry module 88 of IMD 16 (FIG. 4).

Telemetry module 106 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

In some examples, processor 100 of programmer 24 and/or one or more processors of one or more networked computers may perform all or a portion of the techniques described herein with respect to processor 80 and IMD 16. For example, IMD 16 may transmit parameters, such as delivered energy and impedance detected during delivery of a therapeutic electrical signal to patient 14 to programmer 24 via telemetry module 88. Processor 100 and memory 102 may store and process the delivered energy and impedance in the manner described above with reference to processor 80 and memory 82 to identify lead related conditions associated with one or more of leads 18, 20, 22 connected to IMD 16. In other examples, some of the functions associated with identifying lead related conditions may be performed by IMD 16, while others are performed by programmer 24.

FIG. 6 is a block diagram illustrating an example system that includes an external device, such as a server 204, and one or more computing devices 210A-210N, that are coupled to the IMD 16 and programmer 24 shown in FIG. 1 via a network 202. In this example, IMD 16 may use its telemetry module 88 to communicate with programmer 24 via a first wireless connection, and to communication with an access point 200 via a second wireless connection. In the example of FIG. 6, access point 200, programmer 24, server 204, and computing devices 210A-210N are interconnected, and able to communicate with each other, through network 202. In some cases, one or more of access point 200, programmer 24, server 204, and computing devices 210A-210N may be coupled to network 202 through one or more wireless connections. IMD 16, programmer 24, server 204, and computing devices 210A-210N may each comprise one or more processors, such as one or more microprocessors, DSPs, ASICs, FPGAs, programmable logic circuitry, or the like, that may perform various functions and operations, such as those described herein.

Access point 200 may comprise a device that connects to network 202 via any of a variety of connections, such as telephone dial-up, digital subscriber line (DSL), or cable modem connections. In other examples, access point 200 may be coupled to network 202 through different forms of connections, including wired or wireless connections. In some examples, access point 200 may be co-located with patient 14 and may comprise one or more programming units and/or computing devices (e.g., one or more monitoring units) that may perform various functions and operations described herein. For example, access point 200 may include a home-monitoring unit that is co-located with patient 14 and that may monitor the activity of IMD 16. In some examples, server 204 or computing devices 210 may control or perform any of the various functions or operations described herein, e.g., control or assist in performance of integrity tests by IMD 16.

In some cases, server 204 may be configured to provide a secure storage site for archival of, e.g., lead sensing integrity information that has been collected from IMD 16 and/or programmer 24. Network 202 may comprise a local area network, wide area network, or global network, such as the Internet. In some cases, programmer 24 or server 204 may assemble sensing integrity information in web pages or other documents for viewing by and trained professionals, such as clinicians, via viewing terminals associated with computing devices 210. The system of FIG. 6 may be implemented, in some aspects, with general network technology and functionality similar to that provided by the Medtronic CareLink® Network developed by Medtronic, Inc., of Minneapolis, Minn.

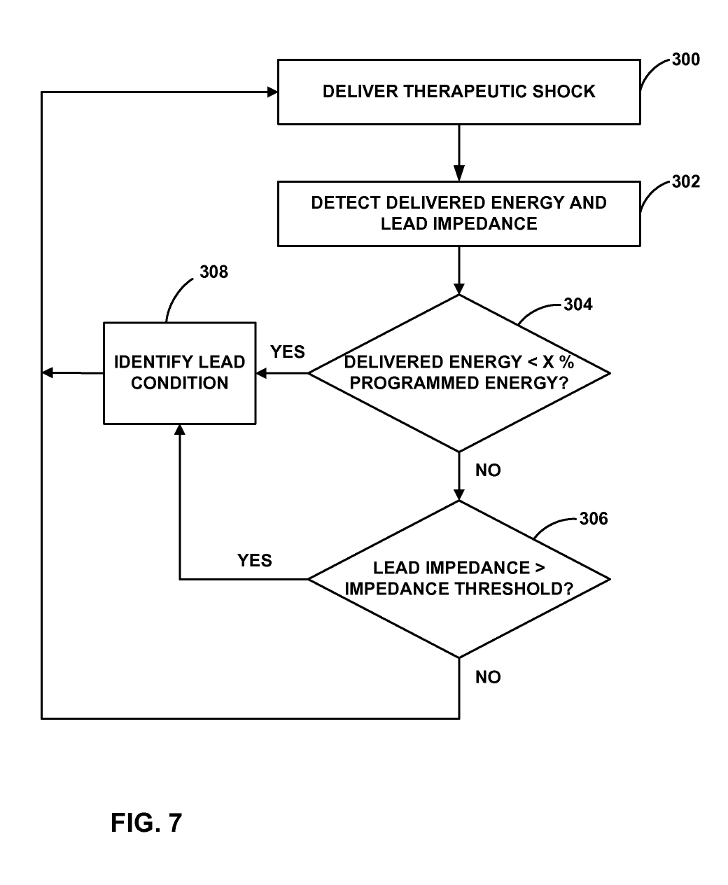

FIG. 7 is a flow diagram of an example method of identifying a lead related condition. The functionality described with respect to FIG. 7 as being provided by a particular processor or device may, in other examples, be provided by any one or more of the processors or devices described herein. For simplicity, the example of FIG. 7 is described in the context of delivering a therapeutic shock including, e.g., a defibrillation or cardioversion shock to patient 14. However, in other examples, the technique of FIG. 7 may be applied to other types of therapeutic electrical signals including, e.g., cardiac pacing pulses. In general, the method illustrated in FIG. 7 includes delivering a therapeutic shock to a patient via a medical lead (300), detecting a delivered energy and an impedance of the lead during the delivery of the shock (302), and comparing the delivered energy detected during the shock to a threshold percentage of a programmed energy of the shock (304) and comparing the detected lead impedance to a threshold impedance value (306). If either the delivered energy is less than the threshold percentage of programmed energy, or the detected impedance is greater than the threshold impedance value, then a lead related condition is identified (308). The lead related condition identification process is repeated anytime a therapeutic shock is delivered to patient 14.

Therapeutic shocks delivered by IMD 16 (300) and during which lead related conditions are identified include, inter alia, pacing pulses configured to capture cardiac tissue, as well as defibrillation shock and cardioversion shocks. In some examples, sensing module 86 of IMD 16 includes narrow band channels with R-wave or P-wave amplifiers that receive signals from different sensing configurations of electrodes 40, 42, 44, 46, 48, 50, 58, 62, 64 or 66, which are used for sensing and/or pacing in right ventricle 28 and/or left ventricle 32 of heart 12. Pacing of heart 12 using signal generator 84 and different combinations of electrodes 40, 42, 44, 46, 48, 50, 58, 62, 64 or 66 may be fixed, rate responsive, and/or in response to a detection of an abnormal rhythm of heart 12 including, e.g., a tachycardia or bradycardia, or unsynchronized left and right ventricular activity targeted by biventricular pacing techniques. In other examples, IMD 16 via signal generator 84 and different combinations of electrodes 58, 62, 64 and 66 delivers cardioversion or defibrillation shocks to patient 14 in response to detecting, e.g., a fibrillation. In some examples, signal generator 84 of IMD 16 delivers a therapeutic shock in the form of a high-voltage pulse between at least two of electrodes 58, 62, 64 or 66. In other examples, signal generator 84 of IMD 16 delivers a therapeutic shock in the form of a high-current pulse between at least two of electrodes 62, 64 or 66.

During delivery of a shock to heart 12 of patient 14, processor 80 automatically detects the energy delivered by and the lead impedance during the delivery of the shock (302). In some examples, processor 80 detects delivered energy and impedance by measuring the actual current delivered through a lead during delivery of a high-voltage pulse. IMD 16, e.g., processor 80, calculates a resistance based upon the voltage amplitude of the pulse and the measured amplitude of the resulting current. Additionally, processor 80 calculates the actual energy delivered to patient 14 by the shock based on the voltage amplitude of the shock, the resulting current, and the time over which the shock was delivered. In other examples, processor 80 detects delivered energy and impedance by measuring the actual voltage delivered through the lead during delivery of a high-current pulse. Processor 80 of IMD 16 calculates a resistance based upon the current amplitude of the pulse and the measured amplitude of the resulting voltage. Additionally, processor 80 calculates the actual energy delivered to patient 14 by the shock based on the current amplitude of the shock, the resulting voltage, and the time over which the shock was delivered.

To identify lead related conditions, processor 80 compares at least one of the detected energy delivered by and the lead impedance during delivery of the therapeutic shock to patient 14 to a threshold. In some examples, processor 80 compares the actual energy delivered by the shock to patient 14 to a threshold percentage of a programmed energy of the shock stored in memory 82 (304). Processor 80 of IMD 16, in general, is programmed to deliver therapy to patient 14 according to one or more programmed parameters organized as therapy programs and program groups and stored in memory 82. One such parameter may include the amount of energy a therapeutic shock is intended to deliver to heart 12 of patient 14. Although signal generator 84, if functioning properly, will generate and deliver the programmed energy in the form of a voltage or current pulse to the electrical path programmed for the shock, the energy actually delivered to patient 14 may be diminished in the event a lead related condition exists somewhere along the electrical path through which the shock is delivered. In order to identify a lead related condition, therefore, processor 80 may determine if the energy delivered to patient 14 by the shock is less than a percentage threshold of the programmed energy of the shock. In some examples, the threshold percentage of programmed energy is in a range from 60 to 80%. In one example, the threshold percentage of programmed energy is approximately equal to 75%.

In other examples, processor 80 compares the lead impedance during delivery of the shock to patient 14 to a threshold impedance value stored in memory 82 (306). In general, it is understood that lead impedance values greater than 200 ohms are indicative of some sort of lead related condition that may compromise the functions of IMD 16. In some examples disclosed herein, the threshold impedance value is in a range from 180 ohms to 220 ohms. In one example, the threshold impedance value is approximately equal to 200 ohms.

If either the delivered energy is less than the threshold percentage of programmed energy, or the detected impedance is greater than the threshold impedance value, then a lead related condition is identified (308). In the event a lead related condition is identified, processor 80 may transmit information regarding such conditions to programmer 24 via telemetry module 88. For example, processor 80 may provide an audible, text based including, e.g., text message or e-mail, or graphical alert regarding any detected lead related conditions, suggest a response to a lead related condition, or provide a detected delivered energy and/or impedance, or other sensed signal for identification of lead related conditions to programmer 24 via telemetry module 88. Processor 80 may also cause IMD 16 to vibrate within patient 14 to alert the patient to detected lead related conditions or cause programmer 24 to vibrate or display a visual alert including, e.g., by emitting light from the programmer. Processor 80 may also receive information regarding lead related conditions or responses to such conditions from programmer 24 via telemetry module 88. In some examples, IMD 16 may signal programmer 24 to further communicate with and pass the alert through a network such as the Medtronic CareLink® Network developed by Medtronic, Inc., of Minneapolis, Minn., or some other network linking patient 14 to a clinician. In another example, IMD 16 may signal programmer 24 to further communicate with and pass the alert to a user through a cellular device, e.g. a cellular telephone. The lead related condition identification process illustrated in FIG. 7 is repeated by IMD 16 anytime a therapeutic shock is delivered to patient 14.

Although detection of lead related conditions is directed herein toward cardiac therapy, this disclosure may also be applicable to other therapies in which detection of lead related conditions may be appropriate. These therapies may include spinal cord stimulation, deep brain stimulation, pelvic floor stimulation, gastric stimulation, occipital stimulation, functional electrical stimulation, and any other stimulation therapy utilizing electrode sensing and/or stimulation methods. Furthermore, although described herein as implemented by an IMD and system including an IMD, in other examples, the techniques described herein may be implemented in an external pulse generator. An external pulse generator may be coupled to leads during implant, and may perform a lead integrity test as described herein to detect any lead related conditions of the recently implanted leads.

In addition, therapy system 10 is not limited to treatment of a human patient. In alternative examples, therapy system 10 may be implemented in non-human patients, e.g., primates, canines, equines, pigs, and felines. These other animals may undergo clinical or research therapies that my benefit from the subject matter of this disclosure.

The techniques described in this disclosure, including those attributed to IMD 16, programmer 24, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the invention defined by the following claims.

1. A method comprising:
delivering a therapeutic electrical signal to a patient via an electrical path including at least one medical lead;
detecting a delivered energy and an impedance of the path during the delivery of the electrical signal; and
identifying a lead related condition when at least one of the detected delivered energy and the detected impendence traverses a respective threshold.

2. The method of claim 1, wherein identifying a lead related condition when at least one of the detected delivered energy and the detected impendence traverses a threshold comprises identifying a lead related condition when both of the detected delivered energy and the detected impendence traverses a respective threshold.

3. The method of claim 1, wherein identifying the lead related condition comprises identifying the lead related condition when the detected energy is less than a percentage threshold of a programmed energy of the electrical signal.

4. The method of claim 3, wherein the percentage threshold of the programmed energy of the electrical signal is in a range from approximately 60% to approximately 80%.

5. The method of claim 4, wherein the percentage threshold of the programmed energy is approximately equal to 75%.

6. The method of claim 1, wherein identifying the lead related condition comprises identifying the lead related condition when the path impedance is greater than a threshold impedance value.

7. The method of claim 6, wherein the threshold impedance value is in a range from approximately 180 ohms to approximately 220 ohms.

8. The method of claim 7, wherein the threshold impedance value is approximately equal to 200 ohms.

9. The method of claim 1, wherein the therapeutic electrical signal comprises at least one of a pacing pulse configured to capture cardiac tissue, a defibrillation shock, or a cardioversion shock.

10. The method of claim 1, wherein the medical lead comprises an implantable medical lead.

11. The method of claim 1, further comprising generating an alert based on the identification of the lead related condition.

12. The method of claim 11, wherein the alert comprises at least one of an audible, text, graphical, device vibration, or light emission alert.

13. The method of claim 11, further comprising at least one of displaying or transmitting the alert.

14. The method of claim 1, further comprising determining a second electrical path configured to deliver the therapeutic electrical signal to the patient.

15. The method of claim 1, further comprising disabling the electrical path through which the therapeutic electrical signal is delivered.

16. A system comprising:
a medical lead;
a signal generator connected to the lead and configured to deliver a therapeutic electrical signal via an electrical path that includes the lead; and
a processor configured to detect a delivered energy and an impedance of the path during delivery of a therapeutic electrical signal to a patient and identify a lead related condition when at least one of the detected delivered energy and the detected impendence traverses a respective threshold.

17. The system of claim 16, wherein the processor identifies a lead related condition when both of the detected delivered energy and the detected impendence traverses a respective threshold.

18. The system of claim 16, wherein the processor identifies a lead related condition when the detected energy is less than a percentage threshold of a programmed energy of the electrical signal.

19. The system of claim 16, wherein the processor identifies a lead related condition when the path impedance is greater than a threshold impedance value.

20. The system of claim 16, wherein the therapeutic electrical signal comprises at least one of a pacing pulse configured to capture cardiac tissue, a defibrillation shock, or a cardioversion shock.

21. The system of claim 16, wherein the lead comprises an implantable medical lead, the system further comprising an implantable medical device that comprises the signal generator and the processor.

22. The system of claim 16, further comprising a programmer that comprises the processor.

23. The system of claim 16, further comprising the processor configured to generate an alert based on the identification of the lead related condition.

24. The system of claim 16, further comprising the processor configured to determine a second electrical path configured to deliver the therapeutic electrical signal to the patient.

25. The system of claim 16, further comprising the processor configured to disable the electrical path through which the therapeutic electrical signal is delivered.

26. A system comprising:
means for delivering a therapeutic electrical signal to a patient via an electrical path;
means for detecting a delivered energy and an impedance of the path during the delivery of the electrical signal; and
means for identifying a lead related condition when at least one of the detected delivered energy and the detected impendence traverses a respective threshold.

27. A computer-readable medium comprising instructions for causing a programmable processor to:
deliver a therapeutic electrical signal to a patient via an electrical path including at least one medical lead;
detect a delivered energy and an impedance of the path during the delivery of the electrical signal; and
identify a lead related condition when at least one of the detected delivered energy and the detected impendence traverses a respective threshold.
